(19) United States
(12) Patent Application Publication
(10) Pub. No.: US 2008/0145433 A1
(43) Pub. Date: Jun. 19, 2008

(54) DARUSENTAN ORAL DOSAGE FORM

(75) Inventors: Michael J. Gerber, Denver, CO (US); Richard J. Gorczynski, Westminster, CO (US); Robert L. Roden, Broomfield, CO (US); Praful K. Shah, Superior, CO (US)

Correspondence Address: HARNESS, DICKEY, & PIERCE, P.L.C, 7700 Bonhomme, Suite 400, ST. LOUIS, MO, 63105, US

(73) Assignee: GILEAD COLORADO, INC., Westminster, CO (US)

(21) Appl. No.: 11/935,474

(22) Filed: Nov. 6, 2007

Related U.S. Application Data
(60) Provisional application No. 60/865,113, filed on Nov. 9, 2006.

(51) Int. Cl. A61K 9/14; A61K 31/44; A61P 3/10

(52) U.S. Cl. 424/489; 514/348

(57) ABSTRACT

A solid discrete orally deliverable pharmaceutical dosage form comprises darusentan and one or more pharmaceutically acceptable excipients; wherein (a) the darusentan is in solid particulate form having a mean particle size of about 5 to about 200 μm and is present in the dosage form in an amount of about 1 to about 600 mg; and (b) the dosage form exhibits at least about 90% dissolution of the darusentan in 30 minutes in a standard in vitro dissolution test. A method for lowering blood pressure, for example in a patient having resistant hypertension, comprises administering such a dosage form once daily to the patient.

This application claims the benefit of U.S. provisional application Ser. No. 60/865,113, filed Nov. 9, 2006, incorporated in its entirety herein by reference.

### FIELD OF THE INVENTION

The present invention relates to orally deliverable pharmaceutical compositions comprising the endothelin receptor antagonist darusentan, and therapeutic methods of use thereof.

### BACKGROUND OF THE INVENTION

Darusentan or LU 135252, (+)-(S)-2-(4,6-dimethoxypyrimidin-2-yloxy)-3-methoxy-3,3-diphenylpropionic acid, and a process for preparing this compound in an enantiomerically pure form, has been disclosed, for example by Riechers et al. (1996) J. Med. Chem. 39:2123-2128. Darusentan has the structure:

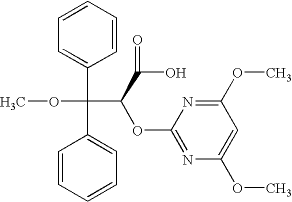

Darusentan is an endothelin-A (ET_{A}) selective receptor antagonist which has been proposed for treatment of hypertension. Endothelin (more particularly the ET-1 isoform thereof) is a small peptide hormone that is believed to play a critical role in control of blood flow and cell growth. Elevated endothelin blood levels are associated with several cardiovascular disease conditions, including pulmonary arterial hypertension, chronic renal disease, coronary artery disease, hypertension, and chronic heart failure. Endothelin is a potent vasoconstrictor, triggering contraction through endothelin-receptor mediated signaling pathways. While antagonism of the ET_{A} receptor is known to reduce endothelin-mediated vasoconstriction, antagonism of the endothelin-B (ET_{B}) receptor can block clearance of ET-1 from the circulatory system, exacerbating its hypertensive effect.

Riechers et al (1996) op. cit. reported that 2-(4,6-dimethoxypyrimidin-2-yloxy)-3-methoxy-3,3-diphenylpropionic acid in racemic form has an affinity for ET_{A} (K_{iA}) of 6 nM and an affinity for ET_{B} (K_{iB}) of 371 nM, thus ET_{A}/ET_{B} selectivity (K_{iB}/K_{iA}) for the racemate based on these data can be calculated as about 62. It was further reported therein that the pure enantiomers have an affinity for ET_{A} of 3 nM and 150 nM. The more potent enantiomer was concluded to be the (S)-enantiomer.

More recently, Lip (2001) IDrugs 4(11):1284-1292 reported that for this (S)-enantiomer, i.e., darusentan, Kᵢ for ET_{A} is 1.4 nM and for ET_{B} is 184 nM, and ET_{A}/ET_{B} selectivity is about 160.

Most antihypertensive drugs are formulated in a dosage form suitable for once daily oral administration. This is an especially convenient dosage interval, and has become standard in the treatment of hypertension. It would be beneficial, therefore, to provide a dosage form of darusentan that exhibits pharmacokinetic properties that, in conjunction with the pharmacodynamic properties of darusentan (in particular its affinity and selectivity for ET_{A}) are consistent with once daily oral administration. It would be especially important to provide such a dosage form where darusentan is to be administered in combination or adjunctive therapy with one or more additional antihypertensive drugs that are formulated for once daily oral administration.

### SUMMARY OF THE INVENTION

There is now provided a solid discrete orally deliverable pharmaceutical dosage form comprising darusentan and one or more pharmaceutically acceptable excipients; wherein (a) the darusentan is in solid particulate form having a mean particle size of about 5 to about 200 μm and is present in the dosage form in an amount of about 1 to about 600 mg; and (b) the excipients are selected and formulated with the darusentan in a manner effective to provide at least about 90% dissolution of the darusentan in 30 minutes when the dosage form is placed in a standard in vitro dissolution test.

There is further provided a tablet having
(i) a core consisting essentially of
   (a) darusentan, about 10 mg to about 100 mg;
   (b) microcrystalline cellulose, about 20 to about 35 mg;
   (c) starch, about 8 to about 12 mg;
   (d) povidone, about 6 to about 12 mg;
   (e) croscarmellose sodium and/or crospovidone, about 2 to about 8 mg total;
   (f) magnesium stearate, about 0.5 to about 1.5 mg;
   (g) colloidal silicon dioxide, about 0.1 to about 1 mg; and
   (h) lactose monohydrate, forming substantially the balance of the core to about 150 to about 200 mg; and
(ii) a film coating, about 5 to about 15 mg;
   or an orally deliverable darusentan composition that is substantially bioequivalent thereto.

There is still further provided a method for lowering blood pressure in a patient, comprising orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a pharmacokinetic profile comprising an average steady-state Cₘᵢₙ/Cₘₐₓ ratio not greater than about 7%, and at least one of
(a) an average Cₘₐₓ of about 30 to about 120 ng/ml per mg darusentan administered;
(b) an average Tₘₐₓ of about 0.5 to about 2 h; and
(c) an average AUC_{0-∞} of about 150 to about 450 ng·h/ml per mg darusentan administered.

Other embodiments, including particular aspects of the embodiments summarized above, will be evident from the detailed description that follows.

### BRIEF DESCRIPTION OF THE DRAWING

FIG. 1 shows the 24-hour time course of plasma concentration of darusentan in subjects receiving 100 mg darusentan in a dosage form of the invention.

### DETAILED DESCRIPTION

The present invention provides orally deliverable pharmaceutical dosage forms comprising darusentan and one or more excipients, and methods of using such dosage forms. In some embodiments, dosage forms useful herein are characterized at least in part by their pharmacokinetic (PK) profile. Parameters forming part of a PK profile include those defined immediately below with particular reference to darusentan.

Cₘₐₓ is the maximum observed or “peak” concentration of darusentan in plasma of a subject following oral administration of a darusentan composition.

Cₘᵢₙ is the minimum observed or “trough” concentration of darusentan in plasma of a subject receiving repeated (e.g., once daily) oral doses of a darusentan composition, and occurs immediately before administration of the next dose. Unless otherwise specified herein, Cₘᵢₙ relates to “steady state”, i.e., after several daily dosing cycles.

Tₘₐₓ is the time to attain Cₘₐₓ following oral administration of a darusentan composition.

T_{1/2} is the half-life of concentration of darusentan in plasma of a subject during a period immediately before administration of the next dose.

AUC (area under the curve) is the product of integration of a plasma concentration/time curve between two defined time points. For example, when considering a once daily dosing interval, AUC₍₀₋₂₄₎ is an AUC value calculated from time 0 (the time of administration) to 24 hours thereafter. AUC_{(0-∞)} is calculated using a conventional pharmacokinetic equation.

It has been discovered in accordance with the present invention that certain darusentan dosage forms have 24-hour efficacy in lowering blood pressure without unacceptable incidence of adverse side effects when orally administered once daily. Such 24-hour efficacy is surprising in view of the PK profile exhibited by these dosage forms. More specifically, the dosage forms in question have an immediate-release PK profile characterized by a high Cₘₐₓ and low Cₘᵢₙ, resulting in a low steady-state Cₘᵢₙ/Cₘₐₓ ratio (not greater than about 7%, in some embodiments not greater than about 5%). For example, a 100 mg darusentan dosage form of the invention exhibited a steady-state Cₘᵢₙ/Cₘₐₓ ratio of 3.8% (see Example 4). In other words, the peak darusentan concentration in plasma following oral administration of this dosage form was over 25 times higher than the trough concentration, reached just prior to the next daily administration.

Such a dosage form would not, a priori, appear to one skilled in the art to be suitable for once a day administration. A sufficient dose must be administered to provide a Cₘᵢₙ consistent with continued high occupancy by darusentan of ET_{A} binding sites. Yet the dose must not be so great as to provide a Cₘₐₓ consistent with substantial occupancy by darusentan of ET_{B} binding sites, as binding of darusentan to ET_{B} can lead to vasoconstriction and other physiological effects that counteract the beneficial effects of ET_{A} binding. Thus, a low steady-state Cₘᵢₙ/Cₘₐₓ ratio, as provided by a dosage form of the present invention, coupled with an ET_{A}/ET_{B} selectivity for darusentan of about 160, as reported for example by Lip (2001), op. cit., gives rise to an expectation of a narrow therapeutic window when darusentan is administered once a day.

It has now been found that the ET_{A}/ET_{B} selectivity of darusentan, when measured in a system that achieves steady-state binding, are much greater than previously reported.

To measure affinities of darusentan for ET_{A} and ET_{B} receptors in the same human tissue preparation, ¹²⁵[I]-endothelin-1 receptor binding cold ligand competition curves were performed in human myocardial membranes prepared from failing and non-failing left ventricles, and cold ligand dissociation constants (Kᵢ) for ET_{A} and ET_{B} receptors were determined by computer modeling. Assay conditions included 10 μM Gpp(NH)p (guanylyl-5′-imidodiphosphate) to eliminate high-affinity agonist binding, 18-point competition curves from 1 pM to 100 μM, and a 4-hour incubation time to achieve steady-state binding. Darusentan was found to have the following properties under these conditions (mean of 8 assays):

| | | |
|---|---|---|
| Kᵢ ET_{A}: | 0.178 ± 0.055 | nM |
| Kᵢ ET_{B}: | 216 ± 85 | nM |

| | |
|---|---|
| ET_{A} selectivity (fold): | 1181 ± 148 |

Without being bound by theory, it is believed that this high affinity and selectivity of darusentan for the ET_{A} receptor, unknown before the present invention, permit a darusentan composition as described herein to be orally administered once daily over a broad range of dosage amounts, resulting in 24-hour antihypertensive efficacy without unacceptable side-effects or reduction in antihypertensive performance related to interaction with ET_{B} around the time of peak plasma concentration of darusentan.

A composition of one embodiment of the invention takes the form of a solid discrete orally deliverable pharmaceutical dosage form comprising darusentan and one or more pharmaceutically acceptable excipients.

Examples of solid discrete orally deliverable dosage forms include compressed or molded tablets, pills and capsules. Typically capsules are solid filled.

In the present embodiment, the darusentan is in solid particulate form having a mean particle size of about 5 to about 200 μm, for example about 10 to about 150 μm or about 25 to about 100 μm. The term “particle size” as used herein refers to size in the longest dimension of the particles. Particle size can be determined on the darusentan component of the composition, most conveniently before mixing with the excipient ingredients of the composition, by any technique known in the art, for example by a laser diffraction technique.

Darusentan can be present in the composition as free acid or as a pharmaceutically acceptable salt thereof, for example an alkali metal salt such as sodium or potassium salts, or an ammonium or organic ammonium salt. Good results have been obtained with darusentan in free acid form and it will generally be found unnecessary to convert it to a salt for preparation of a composition of the present invention. Dosages given herein are expressed on a free acid basis unless the context demands otherwise.

Darusentan is present in the dosage form of the present embodiment in an amount of about 1 mg to about 600 mg, for example about 5 mg to about 450 mg, or about 10 mg to about 300 mg. Generally the amount of darusentan in the dosage form is appropriate for administration of a single dosage form per day to a patient in need thereof. In particular embodiments the amount of darusentan in the dosage form is about 30 mg to about 150 mg, illustratively about 50 mg or about 100 mg.

The dosage form of the present embodiment exhibits at least about 90% dissolution of the darusentan in 30 minutes in a standard in vitro dissolution test using an appropriate dissolution medium. A suitable dissolution test is described in United States Pharmacopeia, 24th ed. (2000) (USP 24) at Test No. 711 thereof entitled “Dissolution”, employing Apparatus No. 2 (“paddle method”). Some dosage forms of the present embodiment can exhibit even faster or more complete dissolution, for example at least about 95% dissolution in 30 minutes.

Unexpectedly, these high rates of dissolution are achieved over a wide range of darusentan particle sizes as set forth above.

In a particular aspect of the present embodiment, a solid discrete orally deliverable pharmaceutical dosage form is provided comprising darusentan and one or more pharmaceutically acceptable excipients; wherein (a) the darusentan is in solid particulate form having a mean particle size of about 25 to about 100 μm and is present in the dosage form in an amount of about 30 to about 150 mg; and (b) the excipients are selected and formulated with the darusentan in a manner effective to provide at least about 95% dissolution of the darusentan in 30 minutes when the dosage form is placed in a standard in vitro dissolution test.

One of ordinary skill in the art will, based on the disclosure herein and without undue experimentation, be able to select suitable excipients and formulate them with darusentan in order to provide a dosage form exhibiting at least about 90%, for example at least about 95%, dissolution of the darusentan in 30 minutes, where the darusentan is in solid particulate form having a mean particle size of about 5 to about 200 μm, for example about 10 to about 150 μm or about 25 to about 100 μm.

A dosage form of the present embodiment typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function. Further information on excipients can be found in standard reference works such as Handbook of Pharmaceutical Excipients, 3rd ed. (Kibbe, ed. (2000), Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

In one aspect, the dosage form is a tablet having a core comprising darusentan and an excipient mixture that comprises
(a) one or more of lactose monohydrate, microcrystalline cellulose and starch;
(b) povidone;
(c) one or more of croscarmellose sodium and crospovidone; and
(d) magnesium stearate;
   and optionally a film coating, in an amount not greater than about 10% by weight of the dosage form, surrounding the core.

Illustratively in such a tablet, the core comprises, by weight thereof,
darusentan, about 5% to about 60%;
lactose monohydrate, about 5% to about 75%;
microcrystalline cellulose, about 10% to about 25%;
starch, about 2% to about 10%;
povidone, about 2% to about 8%;
croscarmellose sodium and/or crospovidone, about 1% to about 5% total;
magnesium stearate, about 0.2% to about 1%; and
colloidal silicon dioxide, zero to about 1%,
   and the film coating, if present, is in an amount of about 2% to about 10% by weight of the core.

In another embodiment of the invention, a pharmaceutical tablet is provided, comprising (i) a core that consists essentially of
darusentan, about 10 mg to about 100 mg;
microcrystalline cellulose, about 20 to about 35 mg;
starch, about 8 to about 12 mg;
povidone, about 6 to about 12 mg;
croscarmellose sodium and/or crospovidone, about 2 to about 8 mg total;
magnesium stearate, about 0.5 to about 1.5 mg;
colloidal silicon dioxide, about 0.1 to about 1 mg; and
lactose monohydrate, forming substantially the balance of the core to about 150 to about 200 mg;
   and (ii) a film coating, about 5 to about 15 mg.

According to this embodiment, mean darusentan particle size is not critical but in one aspect is about 5 to about 200 μm, for example about 10 to about 150 μm or about 25 to about 100 μm. A tablet as just described will generally be found to exhibit at least about 90%, for example at least about 95%, dissolution of the darusentan in 30 minutes in a standard in vitro dissolution test.

Tablets illustrative of this embodiment are described in Example 1 below. Such tablets, when tested in healthy male human volunteers, have PK profiles as shown in Example 2 below.

In a related embodiment of the invention, there is provided an orally deliverable darusentan composition that is substantially bioequivalent to a pharmaceutical tablet as just described, e.g., a tablet as set forth in Example 1. The term “bioequivalent” is defined herein, in general accordance with 21 C.F.R. §320.1, as being not significantly different from such a tablet in the rate and extent to which the active ingredient or active moiety in pharmaceutical equivalents or pharmaceutical alternatives becomes available at the site of drug action when administered at the same molar dose under similar conditions in an appropriately designed study. By “not significantly different” herein is meant having a Cₘₐₓ or AUC that is not less than 80% and not greater than 125% of the Cₘₐₓ or AUC respectively of a tablet as just described.

Any orally deliverable darusentan composition, not restricted to compositions as described with particularity above, meeting the standard of bioequivalence to a tablet as just described, e.g., a tablet as set forth in Example 1, is embraced by the present embodiment. One of ordinary skill in the art, on reading the present disclosure, will be able without undue experimentation to prepare such a composition and submit it for PK testing to determine bioequivalence.

Such a composition can be a solid discrete dosage form such as a tablet, pill or solid-filled or liquid-filled capsule, but alternatively can be non-solid and/or non-discrete, for example in the form of a solution, suspension, elixir, syrup, infusion, powder, granular formulation, etc.

It is not a requirement for bioequivalence of such a composition that it have a Cₘᵢₙ/Cₘₐₓ ratio not greater than about 7%, but optionally it can.

In another embodiment, a dosage form is provided comprising darusentan and one or more pharmaceutically acceptable excipients; wherein (a) the darusentan is in solid particulate form having a mean particle size of about 5 to about 200 μm and is present in the dosage form in an amount of about 1 to about 600 mg; (b) the dosage form exhibits at least about 90% dissolution of the darusentan in 30 minutes in a standard in vitro dissolution test; and (c) upon once daily oral administration to an adult human subject, the dosage form exhibits a PK profile comprising an average steady-state Cₘᵢₙ/Cₘₐₓ, ratio not greater than about 7%, for example not greater than about 5%, and at least one of
(i) an average Cₘₐₓ of about 30 to about 120 ng/ml per mg darusentan administered; and
(ii) an average AUC₀₋₂₄ of about 150 to about 450 ng·h/ml per mg darusentan administered.

Typically, such a dosage form exhibits, as part of its PK profile, an average Tₘₐₓ of about 0.5 to about 2 h.

It has been found in accordance with the present embodiment that Cₘₐₓ and AUC of darusentan, administered in a dosage form as defined above, are related substantially linearly to dose.

In a particular aspect, the PK profile of the dosage form comprises each of
(i) an average Cₘₐₓ of about 30 to about 120 ng/ml per mg darusentan administered;
(ii) an average Tₘₐₓ of about 0.5 to about 2 h; and
(iii) an average AUC₀₋₂₄ of about 150 to about 450 ng·h/ml per mg darusentan administered.

In another particular aspect, the dosage form comprises about 50 mg darusentan, and its PK profile comprises
(i) an average Cₘₐₓ of about 2000 to about 4000 ng/ml;
(ii) an average Tₘₐₓ of about 0.5 to about 2 h; and
(iii) an average AUC₀₋₂₄ of about 9000 to about 18000 ng·h/ml.

In yet another particular aspect, the dosage form comprises about 100 mg darusentan, and its PK profile comprises
(i) an average Cₘₐₓ of about 4000 to about 8000 ng/ml;
(ii) an average Tₘₐₓ of about 0.5 to about 2 h; and
(iii) an average AUC₀₋₂₄ of about 18000 to about 36000 ng·h/ml.

In yet another particular aspect, the dosage form comprises about 150 mg darusentan, and the PK profile comprises
(i) an average Cₘₐₓ of about 6000 to about 12000 ng/ml;
(ii) an average Tₘₐₓ of about 0.5 to about 2 h; and
(iii) an average AUC₀₋₂₄ of about 27000 to about 54000 ng·h/ml.

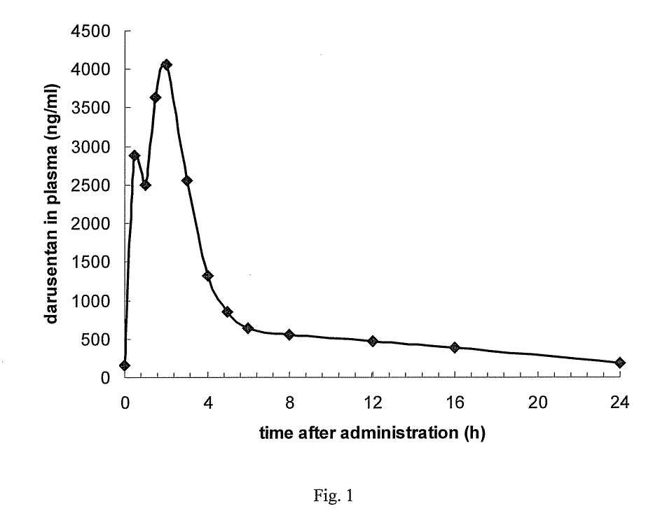

Typically, as illustrated in FIG. 1, the 24-hour time course of darusentan concentration in plasma following oral administration of a dosage form of the invention exhibits a sharp early peak followed by a rapid decline to about 20% of the peak concentration, around 4 to 6 hours after administration. Thereafter, for the remainder of the 24-hour period, further decline is more gradual, consistent with published values of T_{1/2} for darusentan of 10-12 h (see, e.g., Boss et al. (2001) Current Med. Chem. 9:349-383). Accordingly in a further particular aspect, a dosage form of the invention, when orally administered once daily to an adult human subject, exhibits a time course of plasma concentration of darusentan substantially as shown for a 100 mg daily dose in FIG. 1. It will be understood that where the daily dose is greater or less than 100 mg, the absolute levels of darusentan in plasma will be correspondingly higher or lower than shown in FIG. 1; however, the overall shape or profile of the concentration/time curve will be substantially the same. It will further be understood that small perturbations in the curve at specific times, as seen for example at 0.5-1 h in FIG. 1, are not part of the “overall shape or profile” of the curve for the purpose of determining whether a time course of plasma concentration of darusentan is “substantially as shown”.

Adequate characterization of the overall shape or profile of the concentration/time curve typically requires sampling times not only during the first 4 hours after administration but also during the period between 4 and 24 hours after administration. Likewise, a true estimate of AUC, in particular AUC₀₋₂₄, typically requires data points between 4 and 24 hours. It will be understood that for those embodiments herein that are defined in part by AUC, in particular AUC₀₋₂₄, the AUC is determined from a concentration/time curve that includes such data points.

In another embodiment of the present invention, a method is provided for lowering blood pressure in a patient in need thereof. This method comprises orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a PK profile comprising an average steady-state Cₘᵢₙ/Cₘₐₓ ratio not greater than about 7%, and at least one of, for example each of,
(i) an average Cₘₐₓ of about 30 to about 120 ng/ml per mg darusentan administered;
(ii) an average Tₘₐₓ of about 0.5 to about 2 h; and
(iii) an average AUC₀₋₂₄ of about 150 to about 450 ng·h/ml per mg darusentan administered.

A “patient” herein is a human subject in need of treatment to lower blood pressure and/or to mitigate a hypertensive disorder or condition related thereto. Such a patient is typically, but not necessarily, under the care of a medical practitioner, physician or clinician. It is noted that the “adult human subject” in whom the PK profile is determined is generally not the same subject as the patient to be treated, and is usually a healthy subject. It will be understood that standard PK methods, using an appropriate plurality of subjects, should normally be used in determining the PK parameters recited herein, and that mention herein of “an adult human subject” embraces such a plurality of subjects.

It is surprising that a dosage form having such a PK profile can be therapeutically effective for blood pressure reduction when administered once daily. It is especially surprising that, in a particular embodiment, the dosage form having a PK profile as described above can provide a beneficial change in the patient's 24-hour pattern of a blood pressure parameter.

The term “24-hour pattern” in relation to a parameter such as systolic blood pressure (SBP) or diastolic blood pressure (DBP) refers to a cycle in that parameter that recurs approximately daily, for example reflecting underlying endogenous circadian rhythms and/or blood levels of one or more drugs administered in an antihypertensive regimen. For example, increases, decreases, maxima and minima of blood pressure that typically occur each day or night around the same time or times are aspects of the 24-hour pattern. Further aspects include SBP or DBP measured at a specific time in relation to the timing of administration of an antihypertensive drug, for example darusentan. Illustratively, SBP or DBP measured shortly before the regular time of administration is referred to as “trough” SBP or DBP, being measured at a time when levels of the drug circulating in the bloodstream are assumed to be at their lowest. Thus, where the drug is administered once daily at around 8 am, the trough SBP or DBP relates to a blood pressure measurement taken shortly before 8 am on any day. Blood pressure measurements can be recorded in a sitting or reclining subject. In one embodiment, however, 24-hour pattern and effects of the present blood pressure reducing method thereon are established for an ambulatory subject by ambulatory blood pressure (ABP) monitoring.

Examples of beneficial changes in aspects of the 24-hour blood pressure pattern include without limitation
(a) lowering of 24-hour mean ABP;
(b) lowering of trough sitting SBP;
(c) lowering of trough sitting DBP;
(d) lowering of diurnal maximum ABP;
(e) trend away from a bimodal waveform pattern toward a unimodal or less pronouncedly bimodal pattern consistent with normotensive subjects;
(f) increase in day/night ABP ratio; and
(g) at least about 10% nocturnal dipping of ABP.

The present method can lower any one or more measures of blood pressure as described herein, including SBP and/or DBP as determined, for example, by sphygmomanometry. According to certain embodiments, as indicated with particularity hereinbelow, one or more particular measures of blood pressure are specified.

A “trough sitting” SBP or DBP is measured in a sitting subject at a time point when serum concentration of a drug is expected to be at or close to its lowest in a treatment cycle, typically just before administration of a further dose. Illustratively, where the drug or drugs are administered once daily at a particular time, for example around 8 am, trough sitting systolic or diastolic blood pressure can be measured at that time, immediately before the daily administration. It is generally preferred to measure trough sitting SBP or DBP at around the same time of day for each such measurement, to minimize variation due to the natural 24-hour blood pressure cycle.

The course of the 24-hour blood pressure cycle is most conveniently traced by ABP monitoring.

A “24-hour ambulatory” SBP or DBP is an average of measurements taken repeatedly in the course of a 24-hour period, in an ambulatory subject.

A “maximum diurnal” SBP or DBP is a measure of highest SBP or DBP recorded in a 24-hour period, for example by ABP monitoring, and often reflects the peak of the natural 24-hour blood pressure cycle, typically occurring in the morning, for example between about 5 am and about 11 am. Commonly, a second peak occurs in the evening, for example between about 5 pm and 10 pm. Such a bimodal waveform 24-hour ABP pattern may be especially characteristic of resistant hypertension discussed hereinbelow.

Further, a common feature of resistant hypertension is a nighttime, defined herein as 2200 (10 pm) to 0600 (6 am), mean systolic ABP that is no lower, or lower by a margin of less than about 10%, than the daytime, defined herein as 0600 to 2200, mean systolic ABP. The parameter herein termed “day/night ABP ratio” expressed as a percentage is calculated from daytime and nighttime mean systolic ABP using the formula

(daytime mean−nighttime mean)/daytime mean×100.

A 24-hour ABP pattern having a day/night ABP ratio of less than about 10% is sometimes referred to as a “non-dipping ABP.”

Any dose of darusentan that is therapeutically effective, up to a maximum that is tolerated by the patient without unacceptable adverse side effects, can be orally administered according to the present method. For most patients, such a dose is likely to be about 1 to about 600 mg/day, for example about 5 to about 450 mg/day or about 10 to about 300 mg/day. Higher or lower doses can be useful in specific circumstances. In particular embodiments the dose of darusentan administered is about 30 to about 150 mg/day, illustratively about 50 mg/day or about 100 mg/day. The dose is administered once daily but can be administered in one to a plurality of discrete dosage forms, or as a measured quantity of a liquid, powder or granular formulation. In a particular aspect, a single discrete dosage form such as a tablet, pill or capsule provides the daily dose.

In one embodiment of the present method, the composition administered comprises a solid discrete dosage form wherein (a) the darusentan is in solid particulate form having a mean particle size of about 5 to about 200 μm; and (b) the dosage form exhibits at least about 90% dissolution of the darusentan in 30 minutes in a standard in vitro dissolution test. Any such dosage form embraced by the general disclosure herein or more specifically described herein can be used.

A method for lowering blood pressure as described above can be useful in treatment of a hypertensive disorder in the patient. Accordingly, there is still further provided a method for treating a hypertensive disorder in a patient, comprising orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a PK profile comprising an average steady-state Cₘᵢₙ/Cₘₐₓ ratio not greater than about 7%, and at least one of, for example each of,
(i) an average Cₘₐₓ of about 30 to about 120 ng/ml per mg darusentan administered;
(ii) an average Tₘₐₓ of about 0.5 to about 2 h; and
(iii) an average AUC_{0-∞} of about 150 to about 450 ng·h/ml per mg darusentan administered.

Examples of hypertensive disorders that can be treated by the method of this embodiment include conditions marked by systolic hypertension, diastolic hypertension or both, including isolated systolic hypertension and hypertension in the elderly; such conditions can be primary (essential hypertension) or secondary to other conditions including obesity, diabetes, renal disorders (e.g., chronic renal failure, renovascular disease, diabetic nephropathy, etc.), adrenal disorders (e.g., adrenocortical and mineralocorticoid hypertension, pheochromocytoma, primary aldosteronism, Cushing's syndrome, etc.), insulin resistance, salt-sensitivity, polycystic ovary syndrome, sleep apnea, preeclampsia, thyroid and parathyroid diseases, and transplantation. Whether primary or secondary, such hypertension can be, as described above, resistant to baseline antihypertensive therapies, including resistant hypertension and/or refractory hypertension as clinically defined or diagnosed. Hypertensive disorders also include pulmonary arterial hypertension, which likewise can be primary or secondary to various conditions including diseases of the scleroderma spectrum (e.g., mixed connective tissue disease, Raynaud's disease, CREST syndrome, systemic sclerosis, or overlap syndrome); rheumatoid arthritis; chronic hepatitis; systemic lupus erythematosus; anorexigen use; human immunodeficiency virus (HIV) infection; chronic hypoxemia resulting from conditions such as chronic bronchitis, emphysema, sleep apnea, interstitial lung disease, or pulmonary fibrosis; thromboembolic diseases such as in situ thrombosis, tumors, or sickle cell disease; volume and pressure overloads induced primarily from disorders of the left heart (for example, chronic heart failure, septal defects, mitral valve disease, and left atrial myxoma); and disorders directly affecting the pulmonary vasculature such as schistosomiasis, sarcoidosis and pulmonary capillary hemangiomatosis.

The present method can be particularly beneficial where the patient having the hypertensive disorder has a compelling or complicating condition such as diabetes, chronic kidney disease or both.

In yet another embodiment, a method is provided for lowering blood pressure in a patient exhibiting resistance to a baseline antihypertensive therapy with one or more drugs. The method comprises orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a PK profile comprising an average steady-state Cₘᵢₙ/Cₘₐₓ ratio not greater than about 7%, and at least one of, for example each of,
(i) an average Cₘᵢₙ of about 30 to about 120 ng/ml per mg darusentan administered;
(ii) an average Tₘₐₓ of about 0.5 to about 2 h; and
(iii) an average AUC_{0-∞} of about 150 to about 450 ng·h/ml per mg darusentan administered.

A “baseline antihypertensive therapy” herein means a therapeutic regimen comprising administration of one or more drugs, not including darusentan, with an objective (which can be the primary objective or a secondary objective of the regimen) of lowering blood pressure in a hypertensive patient. Each drug according to the regimen is administered at least at a dose considered by an attending physician to be adequate for treatment of hypertension, taking into account the particular patient's medical condition and tolerance for the drug without unacceptable adverse side-effects. An “adequate” dose as prescribed by the physician can be less than or equal to a full dose of the drug. A “full” dose is the lowest of (a) the highest dose of the drug labeled for a hypertension indication; (b) the highest usual dose of the drug prescribed according to JNC 7, BHD-IV, ESH/ESC or WHO/ISH guidelines; or (c) the highest tolerated dose of the drug in the particular patient.

The guidelines referred to above relate to the publications individually cited below and incorporated herein by reference.

JNC 7: Seventh Report of the Joint National Committee on Prevention, Detection, Evaluation, and Treatment of High Blood Pressure (Chobanian et al. (2003) Hypertension 42:1206-1252).

BHD-IV: British Hypertensive Society (J. Human Hypertens. (2004) 18:139-185).

ESH/ESC: European Society of Hypertension/European Society of Cardiology (J. Hypertens. (2003) 21:1011-1053).

WHO/ISH: World Health Organization/International Society of Hypertension (J. Hypertens. (2003) 21:1983-1992).

A baseline antihypertensive therapy illustratively comprises administering one or more diuretics and/or one or more antihypertensive drugs selected from (a) angiotensin converting enzyme (ACE) inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) neutral endopeptidase (NEP) inhibitors, (j) prostanoids, (k) phosphodiesterase type 5 (PDE5) inhibitors, (l) nitrosylated compounds, (m) oral nitrates and (n) inhibitors of renin activity or release. Optionally drugs of still further classes can be included in the baseline therapy, for example to address secondary conditions occurring in a hypertensive patient or side-effects of one or more of the diuretic or antihypertensive drugs.

A patient who is “resistant” to a baseline antihypertensive therapy is one in whom hypertension is failing to respond adequately or at all to the baseline therapy. Typically, the patient receiving the baseline therapy is failing to reach an established blood pressure goal, as set forth for U.S. subjects, for example, in JNC 7 or comparable standards in other countries (e.g., BHD-IV, ESH/ESC or WHO/ISH guidelines). Illustratively, the JNC 7 goal for SBP is<140 mmHg and for DBP<90 mmHg, or for a patient having a complicating condition such as diabetes and/or chronic kidney disease, <130 mmHg SBP and <80 mmHg DBP.

The method of the present invention is especially beneficial where the patient has resistant hypertension (sometimes referred to as refractory hypertension). By definition herein, such a patient exhibits resistance at least to adequate doses of an appropriate three-drug antihypertensive regimen that includes a diuretic. Typically resistant hypertension is diagnosed clinically. In one embodiment, the patient having resistant hypertension exhibits resistance to a baseline antihypertensive therapy that comprises at least the following:
(1) one or more diuretics; and
(2) two or more antihypertensive drugs, selected from at least two of the following classes:
   (a) ACE inhibitors and angiotensin II receptor blockers;
   (b) beta-adrenergic receptor blockers; and
   (c) calcium channel blockers.

In some cases, the patient is resistant to an even more comprehensive baseline therapy, further comprising, for example, one or more direct vasodilators, alpha-1-adrenergic blockers, central alpha-2-adrenergic agonists or other centrally acting antihypertensive drugs, aldosterone receptor antagonists, vasopeptidase inhibitors, NEP inhibitors, prostanoids, PDE5 inhibitors, nitrosylated compounds, oral nitrates or inhibitors of renin activity or release.

Patients resistant to a baseline antihypertensive therapy, especially such a therapy involving a plurality of drugs, clearly represent a very challenging population for treatment. Typically in such patients, increasing dosages of the baseline therapy are not an option because of resulting adverse side effects; furthermore this approach is often ineffective in providing a desired lowering of blood pressure. Accordingly, a darusentan composition as described herein can be orally administered adjunctively with the baseline therapy, optionally modified by dose reduction, or even elimination, of at least one of the drugs in the baseline therapy.

Particularly when used at a full dose, many baseline antihypertensive therapy drugs can have undesirable, in some cases clinically unacceptable or even dangerous, adverse side effects.

For example, especially at full doses, potassium-sparing diuretic drugs can be associated with increased risk of hyperkalemia and related disorders. Overuse of loop diuretics can cause depletion of sodium resulting in hyponatremia and/or extracellular fluid volume depletion associated with hypotension, reduced GRF, circulatory collapse, and thromboembolic episodes. Further, loop diuretics can cause ototoxicity that results in tinnitus, hearing impairment, deafness and/or vertigo. Thiazide diuretics, similarly to loop diuretics, can have adverse effects related to abnormalities of fluid and electrolyte balance. Such adverse events include extracellular volume depletion, hypotension, hypokalemia, hyponatremia, hypochloremia, metabolic alkalosis, hypomagnesemia, hypercalcemia and hyperuricemia. Thiazide diuretics can also decrease glucose tolerance, and increase plasma levels of LDL (low density lipoprotein) cholesterol, total cholesterol, and total triglycerides.

ACE inhibitors are associated with cough and increased risk of angioedema. Beta-adrenergic receptor blockers are associated with increased risk of bronchospasm, bradycardia, heart block, excess negative inotropic effect, peripheral arterial insufficiency and sometimes male impotence. Calcium channel blockers are associated with increased risk of lower limb edema. Further information on adverse events associated with antihypertensive drugs can be found, for example, in standard reference works such as Goodman & Gilman's The Pharmaceutical Basis of Therapeutics, 13th ed. (Brunton et al., eds. (2006), New York: McGraw Hill).

In situations such as those outlined immediately above, optional modification of the baseline therapy by dose reduction or elimination of a baseline therapy drug permitted by oral administration of a darusentan composition as described herein can result in a reduced risk or incidence of adverse events by comparison with the baseline therapy alone without such dose reduction or elimination.

“Adjunctive” administration of the darusentan composition herein means that the darusentan composition is administered concomitantly with one or more additional drugs, in the present instance one or more drugs constituting an optionally modified baseline therapy. For example, the darusentan composition can optionally be administered adjunctively with an adequate to full dose of one or more of the drugs in the baseline therapy, while the other one or more drugs in the baseline therapy are administered at reduced dose or eliminated.

In one aspect of the present embodiment, the dose of the darusentan composition administered is effective, for example in combination with the optionally modified baseline therapy, to provide a reduction of at least about 3 mmHg in trough sitting SBP and/or DBP, 24-hour ambulatory SBP and/or DBP, and/or maximum diurnal SBP and/or DBP.

In a particular aspect, the patient has resistant systolic hypertension, and the dose of the darusentan composition administered is effective, for example in combination with the optionally modified baseline therapy, to provide a reduction of at least about 3 mmHg in one or more of trough sitting, 24-hour ambulatory and maximum diurnal SBP.

In an even more particular aspect, the at least about 3 mmHg reduction is observed in trough sitting SBP, and at least comparable reductions can be, but are not necessarily, observable in 24-hour ambulatory and/or maximum diurnal SBP. In some cases the method is effective to provide a greater reduction in trough sitting SBP, for example a reduction of at least about 5 mmHg, at least about 7 mmHg or at least about 10 mmHg.

The present method can increase the likelihood of a patient achieving SBP goal, for example a JNC 7, BHD-IV, ESH/ESC or WHO/ISH goal for SBP. Thus in a still further particular aspect, a JNC 7 goal for SBP is achieved, for example a trough sitting or 24-hour ambulatory SBP of <140 mmHg or, in the case of a patient with diabetes or chronic kidney disease, <130 mmHg.

In another particular aspect, the patient has resistant diastolic hypertension, and the dose and frequency of the administration of darusentan and the inhibitor of renin activity or release is effective, for example in combination with the optionally modified baseline therapy, to provide a reduction of at least about 3 mmHg in one or more of trough sitting, 24-hour ambulatory and maximum diurnal DBP.

In an even more particular aspect, the at least about 3 mmHg reduction is observed in trough sitting DBP, and at least comparable reductions can be, but are not necessarily, observable in 24-hour ambulatory and/or maximum diurnal DBP. In some cases the method is effective to provide a greater reduction in trough sitting DBP, for example a reduction of at least about 5 mmHg, at least about 7 mmHg or at least about 10 mmHg.

The present method can increase the likelihood of a patient achieving DBP goal, for example a JNC 7, BHD-IV, ESH/ESC or WHO/ISH goal for DBP. Thus in a still further particular aspect, a JNC 7 goal for DBP is achieved, for example a trough sitting or 24-hour ambulatory DBP of <90 mmHg or, in the case of a patient with diabetes or chronic kidney disease, <80 mmHg.

In yet another aspect, administration of a darusentan composition as described herein to a patient having resistant hypertension is effective to provide a beneficial change in the patient's 24-hour pattern of SBP and/or DBP. Examples of the kinds of beneficial changes are listed hereinabove.

Because of the particular criticality of controlling blood pressure in patients with complicating conditions such as diabetes and/or chronic kidney disease, and the greater difficulty of lowering blood pressure to the lower levels consistent with good management of these conditions, administration of a darusentan composition as described herein can be especially beneficial for such patients.

In a particular aspect, a darusentan composition as described herein is administered to a patient having resistant hypertension and a compelling or complicating condition such as diabetes, chronic kidney disease or both.

In yet another embodiment, a method is provided for providing a beneficial effect on renal and/or cardiovascular function in a patient having resistant hypertension. The method comprises orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a PK profile comprising an average steady-state Cₘᵢₙ/Cₘₐₓ ratio not greater than about 7%, and at least one of, for example each of,
(i) an average Cₘₐₓ of about 30 to about 120 ng/ml per mg darusentan administered;
(ii) an average Tₘₐₓ of about 0.5 to about 2 h; and
(iii) an average AUC_{0-∞} of about 150 to about 450 ng·h/ml per mg darusentan administered.

“Providing a beneficial effect” in the present context includes enhancing, maintaining or moderating a decline in renal or cardiovascular function, and also includes preventing one or more cardiovascular adverse events. Optionally, the darusentan composition can be administered adjunctively, or in combination, with one or more additional drugs as described herein.

In one aspect of this embodiment, the beneficial effect comprises preventing one or more cardiovascular adverse events. Examples of cardiovascular adverse effects include without limitation acute coronary syndrome (including unstable angina and non-Q wave infarction), myocardial infarction, heart failure, systolic heart failure, diastolic heart failure (also known as diastolic dysfunction), stroke, occlusive stroke, hemorrhagic stroke and combinations thereof. “Preventing” in the present context includes reducing risk, incidence and/or severity of a subsequent cardiovascular adverse effect. Optionally, the darusentan composition can be administered adjunctively, or in combination, with one or more additional drugs as described herein.

In another aspect of the present embodiment, a beneficial effect is obtained on renal function. Such an effect can be observed, for example, by monitoring one or more blood and/or urinary biomarkers. Examples of such biomarkers include without limitation serum creatinine, serum insulin, serum glutamic acid decarboxylase (GAD), serum protein tyrosine phosphatase-like molecule IA2, blood urea nitrogen, urinary protein, urinary albumin, microalbuminuria, urinary β2-microglobulin, urinary N-acetyl-β-glucosaminidase, urinary retinol binding protein, urinary sodium, glomerular filtration rate, urinary albumin to creatinine ratio, urine volume, and combinations thereof.

Illustratively, the darusentan composition can be administered at a dose effective to lower urinary albumin to creatinine ratio. This can be especially beneficial where the baseline urinary albumin to creatinine ratio is greater than about 30 mg/g or where baseline 24-hour urinary albumin is greater than about 30 mg/day.

Variants and illustrative modalities of each of the methods described herein, for example beneficial changes obtained, dosages, formulations, and optional additional diuretics and/or additional antihypertensive drugs are as described hereinabove. Thus any darusentan composition exhibiting, upon once daily oral administration to an adult human subject, a PK profile comprising an average steady-state Cₘᵢₙ/Cₘₐₓ ratio not greater than about 7%, and at least one of, for example each of,
(i) an average Cₘₐₓ of about 30 to about 120 ng/ml per mg darusentan administered;
(ii) an average Tₘₐₓ of about 0.5 to about 2 h; and
(iii) an average AUC_{0-∞} of about 150 to about 450 ng·h/ml per mg darusentan administered.
   illustratively but without limitation a dosage form as described generally or with particularity herein, may be found suitable for administration according to the present methods.

### EXAMPLES

### Example 1

### Darusentan Film-Coated Tablets

Darusentan 10 mg, 50 mg and 100 mg tablets, and placebo tablets containing no darusentan, were prepared having compositions as shown in Table 1.

TABLE 1
Tablet compositions

| | Amount (mg/tablet) | | | |
|---|---|---|---|---|
| | placebo | 10 mg | 50 mg | 100 mg |
| Ingredient | tablet | tablet | tablet | tablet |

CORE:

| | | | | |
|---|---|---|---|---|
| darusentan | | 10.0 | 50.0 | 100.0 |
| lactose monohydrate | 157.3 | 108.0 | 68.0 | 18.0 |
| corn starch | | 10.0 | 10.0 | 10.0 |
| microcrystalline cellulose 102 | | 27.1 | 27.1 | 27.1 |
| povidone K30 | 5.9 | 8.5 | 8.5 | 8.5 |
| croscarmellose sodium | | 5.0 | 5.0 | 5.0 |
| magnesium stearate | 0.9 | 0.9 | 0.9 | 0.9 |
| colloidal silicon dioxide | | 0.5 | 0.5 | 0.5 |
| crospovidone | 5.9 | 0 | 0 | 0 |
| total core | 170.0 | 170.0 | 170.0 | 170.0 |

COATING:

| | | | | |
|---|---|---|---|---|
| talc | 3.87 | 3.87 | 3.87 | 3.87 |
| titanium dioxide E171 | 2.84 | 2.84 | 2.84 | 2.84 |
| hypromellose 2910 | 2.26 | 2.26 | 2.26 | 2.26 |
| PEG 6000 | 0.39 | 0.39 | 0.39 | 0.39 |
| PEG 400 | 0.58 | 0.58 | 0.58 | 0.58 |
| red iron oxide E172 | 0.06 | 0.06 | 0.06 | 0.06 |
| total coating | 10.0 | 10.0 | 10.0 | 10.0 |

Darusentan used in preparing the 10 mg, 50 mg and 100 mg tablets had a mean particle size in the range of about 25 to about 100 μm.

### Example 2

### PK of Darusentan Tablets Following Single Oral Dose Administration

A trial was conducted to investigate, inter alia, pharmacolinetics of darusentan in 25 healthy adult male volunteers between 18 and 45 years of age. This was a randomized, double-blind, alternating panel, single ascending dose trial with repeated measurements and intra-individual placebo control; the treatments (dose levels of darusentan and placebo) were unbalanced. Darusentan was administered in a single dose of 5 mg (n=6), 10 mg (n=20), 15 mg (n=6), 20 mg (n=6), 25 mg (n=6), 30 mg (n=7), 50 mg (n=6), 75 mg (n=5), 100 mg (n=5) and 150 mg (n=5).

Placebo, 10 mg and 100 mg tablets, substantially as prepared in Example 1, were used in the study. Darusentan used in preparing the 10 mg and 100 mg tablets had a mean particle size in the range of about 25 to about 100 μm. Tablets were halved if necessary to provide the full range of doses.

In a standard in vitro dissolution test of 6 replicates using the paddle method, each of the 10 mg and 100 mg tablet formulations exhibited substantially complete dissolution of darusentan (mean calculated dissolution 99% and 101% respectively) in 30 minutes.

Blood sampling for determination of plasma concentrations of darusentan was done prior to (0 h) and at 0.5, 1, 1.5, 2, 3, 4, 5, 6, 8, 12, 16, 24, 36 and 48 h after administration of the tablets.

Arithmetic means and standard deviations of Cₘₐₓ and AUC_{0-∞}, and median values and ranges of Tₘₐₓ, based on individual values of these parameters calculated by standard PK methods, are presented in Tables 2A and 2B. As this was a single dose study, Cₘᵢₙ was not determined.

TABLE 2A
PK parameters from single dose oral administration of darusentan tablets (5-25 mg)

| | Dose | | | | |
|---|---|---|---|---|---|
| PK Parameter | 5 mg | 10 mg | 15 mg | 20 mg | 25 mg |
| Cₘₐₓ (ng/ml) | 227 ± 55 | 728 ± 233 | 1034 ± 138 | 1410 ± 379 | 1441 ± 491 |
| Tₘₐₓ (h) | 1.0 | 1.0 | 0.75 | 1.0 | 1.25 |
| | (0.5-1.0) | (0.5-3.0) | (0.5-1.0) | (0.5-1.5) | (0.5-3.0) |
| AUC_{0-∞} (ng · h/ml) | 1007 ± 163 | 2874 ± 865 | 4084 ± 956 | 4800 ± 1281 | 6084 ± 1715 |

TABLE 2B
PK parameters from single dose oral administration of darusentan tablets (30-150 mg)

| | Dose | | | | |
|---|---|---|---|---|---|
| PK Parameter | 30 mg | 50 mg | 75 mg | 100 mg | 150 mg |
| Cₘₐₓ (ng/ml) | 1698 ± 465 | 3198 ± 1084 | 3965 ± 1854 | 5694 ± 1462 | 8372 ± 1569 |
| Tₘₐₓ (h) | 1.0 | 1.25 | 1.0 | 1.0 | 1.5 |
| | (0.5-3.0) | (0.5-2.0) | (0.5-3.0) | (0.5-1.5) | (0.5-3.0) |
| AUC_{0-∞}(ng · h/ml) | 7179 ± 1809 | 14326 ± 4170 | 18137 + 5556 | 25102 ± 3552 | 43674 ± 18720 |

Cₘₐₓ and AUC_{0-∞} increased with the dose. The slopes of individual log/log regression lines for individual subjects were scattered around 1 (0.62 to 1.20 for Cₘₐₓ, 0.83 to 1.16 for AUC_{0-∞}) and, in their entirety, showed dose linearity with regard to these PK parameters. In a pooled analysis over all subjects a significant deviation from dose linearity was found neither for Cₘₐₓ nor for AUC_{0-∞}.

### Example 3

### PK of Darusentan Tablets Following Single Oral Dose Administration

A further trial was conducted to investigate, inter alia, pharmacokinetics of darusentan in 95 (9 female, 86 male) congestive heart failure (CHF) patients. This was a multi-center, prospective, uncontrolled clinical trial. Five doses (1 mg, 10 mg, 30 mg, 100 mg and 300 mg) of darusentan were studied consecutively. At least 12 patients received each dose, in addition to their normal medication for CHF. Pharmacokinetics were not determined for the 1 mg dose of darusentan.

Darusentan 1 mg, 10 mg, 30 mg, 100 mg and 300 mg tablets, generally similar to those of Example 1 but uncoated, were used in the study.

Blood sampling for determination of plasma concentrations of darusentan was done prior to (0 h) and at 0.5, 1, 1.5, 2, 3, 4 and 24 h after administration of the tablets.

Arithmetic means and standard deviations of Cₘₐₓ, Tₘₐₓ and AUC, based on individual values of these parameters calculated by standard PK methods, are presented in Table 3. As this was a single dose study, Cₘᵢₙ was not determined.

TABLE 3
PK parameters from single dose oral administration of darusentan tablets

| | Dose | | | |
|---|---|---|---|---|
| PK Parameter | 10 mg | 30 mg | 100 mg | 300 mg |
| Cₘₐₓ (ng/ml) | 615 ± 151 | 1765 ± 470 | 6531 ± 2033 | 16767 ± 5103 |
| Tₘₐₓ (h) | 2.1 ± 0.7 | 1.8 ± 0.8 | 1.9 ± 0.8 | 2.4 ± 0.9 |
| AUC (ng · h/ml) | 4373 ± 1598 | 12666 ± 7342 | 49982 ± 31007 | 137404 ± 54742 |

Pharmacokinetics of darusentan again appeared to be linear, as both Cₘₐₓ and AUC increased proportionally with the dose. The values of AUC calculated from the data in this study may be unrepresentatively high, due to the absence of plasma concentration data between 4 h and 24 h after administration.

### Example 4

### PK of Darusentan Tablets Following Single Oral Dose Administration

A trial was conducted to investigate, inter alia, pharmacokinetics of darusentan in healthy adult male volunteers between 18 and 45 years of age. This was a randomized, double-blind, multiple ascending dose, parallel group design trial with inter-individual placebo control; the treatments (dose levels of darusentan and placebo) were unbalanced. Darusentan was administered in ascending dose steps of 10 mg, 30 mg, 60 mg and 100 mg. Each dose was administered once daily for 6 days.

Placebo, 10 mg and 100 mg tablets, substantially as prepared in Example 1, were used in the study. Darusentan used in preparing the 10 mg and 100 mg tablets had a mean particle size in the range of about 25 to about 100 μm.

Blood sampling for determination of plasma concentrations of darusentan was done prior to (0 h) administration of the tablets on each of days 1, 3, 4, 5 and 6. On days 1 and 6 only, blood sampling for determination of plasma concentrations of darusentan was done at 0.5, 1, 1.5, 2, 3, 4, 5, 6, 8, 12, 16 and 24, and (on day 6 only) 36 and 48 h after administration of the tablets.

Arithmetic means and standard deviations of Cₘₐₓ, Tₘₐₓ, Cₘᵢₙ and AUC₀₋₂₄, based on individual values of these parameters calculated by standard PK methods, are presented for day 1 in Table 4 and for day 6 in Table 5. In each case a value of Cₘᵢₙ/Cₘₐₓ, calculated from the mean values of Cₘᵢₙ and Cₘₐₓ, is also shown.

TABLE 4
PK parameters from once daily oral administration
of darusentan tablets (day 1)

| PK | Dose | | | |
|---|---|---|---|---|
| Parameter | 10 mg | 30 mg | 60 mg | 100 mg |
| Cₘₐₓ | 606 ± 181 | 1470 ± 416 | 2652 ± 989 | 5667 ± 1053 |
| (ng/ml) | | | | |
| Tₘₐₓ | 1.4 ± 0.8 | 1.4 ± 0.8 | 1.1 ± 0.6 | 1.4 ± 0.6 |
| (h) | | | | |
| Cₘᵢₙ | 17 ± 5 | 40 ± 12 | 94 ± 64 | 168 ± 73 |
| (ng/ml) | | | | |
| Cₘᵢₙ/ | 2.8 | 2.7 | 3.5 | 3.0 |
| Cₘₐₓ (%) | | | | |
| AUC₀₋₂₄ | 2087 ± 689 | 6079 ± 878 | 12136 ± 4875 | 22530 ± 7948 |
| (ng · h/ml) | | | | |

TABLE 5
PK parameters from once daily oral administration
of darusentan tablets (day 6)

| PK | Dose | | | |
|---|---|---|---|---|
| Parameter | 10 mg | 30 mg | 60 mg | 100 mg |
| Cₘₐₓ | 619 ± 189 | 1786 ± 134 | 2657 ± 982 | 4867 ± 1084 |
| (ng/ml) | | | | |
| Tₘₐₓ | 0.7 ± 0.4 | 0.8 ± 0.2 | 1.8 ± 0.9 | 1.5 ± 0.3 |
| (h) | | | | |
| Cₘᵢₙ | 24 ± 3 | 82 ± 19 | 139 ± 44 | 184 ± 41 |
| (ng/ml) | | | | |
| Cₘᵢₙ/ | 3.9 | 4.6 | 5.2 | 3.8 |
| Cₘₐₓ (%) | | | | |
| AUC₀₋₂₄ | 2174 ± 178 | 6792 ± 426 | 13730 ± 2717 | 19758 ± 2192 |
| (ng · h/ml) | | | | |

Day 6 data, representing an approach to steady state with regard to Cₘᵢₙ, showed that in general, across doses, the Cₘᵢₙ/Cₘₐₓ ratio was about 3% to about 5%.

FIG. 1 shows the time course of plasma concentration of darusentan in subjects receiving the 100 mg dose (average of 4 subjects, day 6). Lower doses exhibited a similar time course (not shown).

All patents and publications cited herein are incorporated by reference into this application in their entirety.

The words “comprise”, “comprises”, and “comprising” are to be interpreted inclusively rather than exclusively.

What is claimed is:

1. A solid discrete orally deliverable pharmaceutical dosage form comprising darusentan and one or more pharmaceutically acceptable excipients; wherein (a) the darusentan is in solid particulate form having a mean particle size of about 5 to about 200 μm and is present in the dosage form in an amount of about 1 to about 600 mg; and (b) the excipients are selected and formulated with the darusentan in a manner effective to provide at least about 90% dissolution of the darusentan in 30 minutes when the dosage form is placed in a standard in vitro dissolution test.

2. The dosage form of claim 1, having a mean darusentan particle size of about 25 to about 100 μm.

3. The dosage form of claim 1, comprising about 5 to about 300 mg darusentan.

4. The dosage form of claim 1, comprising about 10 to about 150 mg darusentan.

5. The dosage form of claim 1, exhibiting at least about 95% dissolution of the darusentan in 30 minutes in a standard in vitro dissolution test.

6. The dosage form of claim 1, wherein upon once daily oral administration of the dosage form to an adult human subject a pharmacokinetic profile is obtained that comprises an average steady-state Cₘᵢₙ/Cₘₐₓ ratio not greater than about 7%.

7. The dosage form of claim 6, wherein the average steady-state Cₘᵢₙ/Cₘₐₓ ratio is not greater than about 5%.

8. The dosage form of claim 6, wherein the pharmacokinetic profile further comprises at least one of
(a) an average Cₘₐₓ of about 30 to about 120 ng/ml per mg darusentan administered;
(b) an average Tₘₐₓ of about 0.5 to about 2 h; and
(c) an average AUC₀₋₂₄ of about 150 to about 450 ng·h/ml per mg darusentan administered.

9. The dosage form of claim 6, wherein the pharmacokinetic profile further comprises each of
(a) an average Cₘₐₓ of about 30 to about 120 ng/ml per mg darusentan administered;
(b) an average Tₘₐₓ of about 0.5 to about 2 h; and
(c) an average AUC₀₋₂₄ of about 150 to about 450 ng·h/ml per mg darusentan administered.

10. The dosage form of claim 6, comprising about 50 mg darusentan, wherein the pharmacokinetic profile comprises
(a) an average Cₘₐₓ of about 2000 to about 4000 ng/ml;
(b) an average Tₘₐₓ of about 0.5 to about 2 h; and
(c) an average AUC₀₋₂₄ of about 9000 to about 18000 ng·h/ml.

11. The dosage form of claim 6, comprising about 100 mg darusentan, wherein the pharmacokinetic profile comprises
(a) an average Cₘₐₓ of about 4000 to about 8000 ng/ml;
(b) an average Tₘₐₓ of about 0.5 to about 2 h; and
(c) an average AUC₀₋₂₄ of about 18000 to about 36000 ng·h/ml.

12. The dosage form of claim 6, comprising about 150 mg darusentan, wherein the pharmacolidnetic profile comprises
(a) an average Cₘₐₓ of about 6000 to about 12000 ng/ml;
(b) an average Tₘₐₓ of about 0.5 to about 2 h; and
(c) an average AUC₀₋₂₄ of about 27000 to about 54000 ng·h/ml.

13. The dosage form of claim 1 that, when orally administered once daily to an adult human subject, exhibits a time course of plasma concentration of darusentan substantially as shown in FIG. 1.

14. The dosage form of claim 1, in a form of a tablet.

15. The dosage form of claim 14, wherein the excipient(s) comprise one or more materials independently selected from the group consisting of diluents, binding agents, disintegrants and antifrictional agents.

16. The dosage form of claim 15, wherein the excipient(s) comprise one or more diluents independently selected from the group consisting of lactose, lactitol, maltitol, mannitol, sorbitol, xylitol, dextrose, fructose, sucrose, sucrose-based diluents, maltose, inositol, hydrolyzed cereal solids, starch, amylose, dextrates, pregelatinized starch, dextrins, powdered cellulose, microcrystalline cellulose, silicified microcrystalline cellulose, cellulose acetate, calcium salts, magnesium carbonate, magnesium oxide, bentonite, kaolin and sodium chloride.

17. The dosage form of claim 15, wherein the excipient(s) comprise one or more binding agents independently selected from the group consisting of acacia, tragacanth, glucose, polydextrose, starch, pregelatinized starch, gelatin, methylcellulose, carmellose sodium, hydroxypropylmethylcellulose, hydroxypropylcellulose, hydroxyethylcellulose, ethylcellulose, dextrins, zein, alginic acid, alginates, magnesium aluminum silicate, bentonite, polyethylene glycol, polyethylene oxide, guar gum, polysaccharide acids, povidone, carbomers and polymethacrylates.

18. The dosage form of claim 15, wherein the excipient(s) comprise one or more disintegrants independently selected from the group consisting of starch, pregelatinized starch, sodium starch glycolate, clays, magnesium aluminum silicate, powdered cellulose, microcrystalline cellulose, methylcellulose, low-substituted hydroxypropyl-cellulose, carmellose, carmellose calcium, croscarmellose sodium, alginates, povidone, crospovidone, polacrilin potassium, gums and colloidal silicon dioxide.

19. The dosage form of claim 15 wherein the excipient(s) comprise one or more antifrictional agents independently selected from the group consisting of glyceryl behenate, stearic acid, metallic stearates, hydrogenated vegetable oils, glyceryl palmitostearate, talc, waxes, sodium benzoate, sodium acetate, sodium fumarate, sodium stearyl fumarate, polyethylene glycol, poloxamers, polyvinyl alcohol, sodium oleate, sodium lauryl sulfate, magnesium lauryl sulfate, colloidal silicon dioxide, starch, DL-leucine, powdered cellulose and magnesium trisilicate.

20. The dosage form of claim 14, having a composition adapted for direct compression.

21. The dosage form of claim 14, having a core comprising darusentan and an excipient mixture that comprises
(a) one or more of lactose monohydrate, microcrystalline cellulose and starch;
(b) povidone;
(c) one or more of croscarmellose sodium and crospovidone; and
(d) magnesium stearate;
and optionally a film coating, in an amount not greater than about 10% by weight of the dosage form, surrounding the core.

22. The dosage form of claim 21, wherein the core comprises, by weight thereof,
darusentan, about 5% to about 60%;
lactose monohydrate, about 5% to about 75%;
microcrystalline cellulose, about 10% to about 25%;
starch, about 2% to about 10%;
povidone, about 2% to about 8%;
croscarmellose sodium and/or crospovidone, about 1% to about 5% total;
magnesium stearate, about 0.2% to about 1%; and
colloidal silicon dioxide, zero to about 1%.

23. The dosage form of claim 22, further comprising a film coating, in an amount of about 2% to about 10% by weight of the core.

24. The dosage form of claim 23, having a core consisting essentially of
darusentan, about 10 mg to about 100 mg;
microcrystalline cellulose, about 20 to about 35 mg;
starch, about 8 to about 12 mg;
povidone, about 6 to about 12 mg;
croscarmellose sodium and/or crospovidone, about 2 to about 8 mg total;
magnesium stearate, about 0.5 to about 1.5 mg;
colloidal silicon dioxide, about 0.1 to about 1 mg;
lactose monohydrate, forming substantially the balance to about 150 to about 200 mg;
and a film coating, about 5 to about 15 mg.

25. An orally deliverable darusentan composition that is substantially bioequivalent to the dosage form of claim 24.

26. A method for lowering blood pressure, comprising orally administering once daily to a patient in need thereof a pharmaceutical composition comprising darusentan in an amount of about 1 to about 600 mg and at least one pharmaceutically acceptable excipient; wherein the composition, upon once daily oral administration to an adult human subject, exhibits a pharmacokinetic profile comprising an average Cₘᵢₙ/Cₘₐₓ ratio not greater than about 7%, and at least one of
(a) an average Cₘₐₓ of about 30 to about 120 ng/ml per mg darusentan administered;
(b) an average Tₘₐₓ of about 0.5 to about 2 h; and
(c) an average AUC_{0-∞} of about 150 to about 450 ng·h/ml per mg darusentan administered.

27. The method of claim 26, comprising administering a composition that comprises a solid discrete dosage form wherein (a) the darusentan is in solid particulate form having a mean particle size of about 5 to about 200 μm; and (b) the dosage form exhibits at least about 90% dissolution of the darusentan in 30 minutes in a standard in vitro dissolution test.

28. The method of claim 26, wherein a beneficial change is provided in the patient's 24-hour pattern of a blood pressure parameter.

29. The method of claim 26, wherein the patient exhibits resistance to a baseline antihypertensive therapy with one or more drugs.

30. The method of claim 29, wherein the patient has resistant hypertension.

31. A method for treating a hypertensive disorder in a patient, the method comprising orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a pharmacolinetic profile comprising an average Cₘᵢₙ/Cₘₐₓ ratio not greater than about 7%, and at least one of
(a) an average Cₘₐₓ of about 30 to about 120 ng/ml per mg darusentan administered;
(b) an average Tₘₐₓ of about 0.5 to about 2 h; and
(c) an average AUC_{0-∞} of about 150 to about 450 ng·h/ml per mg darusentan administered.

32. The method of claim 31, comprising administering a composition that comprises a solid discrete dosage form wherein (a) the darusentan is in solid particulate form having a mean particle size of about 5 to about 200 μm; and (b) the dosage form exhibits at least about 90% dissolution of the darusentan in 30 minutes in a standard in vitro dissolution test.

33. The method of claim 31, wherein the hypertensive disorder comprises one or more conditions selected from the group consisting of systolic hypertension; diastolic hypertension; isolated systolic hypertension; hypertension in the elderly; essential hypertension; hypertension secondary to obesity, diabetes, renal disorders, adrenal disorders, insulin resistance, salt-sensitivity, polycystic ovary syndrome, sleep apnea, preeclampsia, thyroid and parathyroid diseases, and transplantation; resistant hypertension; and pulmonary arterial hypertension.

34. The method of claim 31, wherein the patient has diabetes, chronic kidney disease or both.

35. A method for providing a beneficial effect on renal and/or cardiovascular function in a patient having resistant hypertension, the method comprising orally administering once daily to the patient a pharmaceutical composition comprising darusentan in an amount of about 1 to about 600 mg and at least one pharmaceutically acceptable excipient; wherein the composition, upon once daily oral administration to an adult human subject, exhibits a pharmacokinetic profile comprising an average Cₘᵢₙ/Cₘₐₓ ratio not greater than about 7%, and at least one of
(a) an average Cₘₐₓ of about 30 to about 120 ng/ml per mg darusentan administered;
(b) an average Tₘₐₓ of about 0.5 to about 2 h; and
(c) an average AUC_{0-∞} of about 150 to about 450 ng·h/ml per mg darusentan administered.

36. The method of claim 35, comprising administering a composition that comprises a solid discrete dosage form wherein (a) the darusentan is in solid particulate form having a mean particle size of about 5 to about 200 μm; and (b) the dosage form exhibits at least about 90% dissolution of the darusentan in 30 minutes in a standard in vitro dissolution test.
